(19) United States
(12) Patent Application Publication
(10) Pub. No.: US 2005/0048113 A1
(43) Pub. Date: Mar. 3, 2005

(54) Composition and method for treatment or prevention of oral cavity disorders

(76) Inventor: Muhammad Abdulrazik, East Jerusalem (IL)

Correspondence Address: Shiboleth, Yisraeli, Roberts, Zisman & Co.;60th Floor, 350 Fifth Avenue, New York, NY, 10118, US

(21) Appl. No.: 10/931,289

(22) Filed: Sep. 1, 2004

(30) Foreign Application Priority Data
Sep. 2, 2003 (IL) 157707

(51) Int. Cl. A61K 9/20; A61K 33/06

(52) U.S. Cl. 424/464; 514/338; 424/682

(57) ABSTRACT

A method for treatment or prevention of oral cavity diseases and disorders and/or related states, in a subject in need of such treatment, comprising local administration of antisecretory drugs and/or antacids, in acceptable pharmacological forms and pharmaceutical formulations.

### FIELD OF THE INVENTION

The present invention relates generally to the field of prevention or treatment of oral cavity diseases and disorders, and more particularly, the present invention relates to a method for prevention or treatment of oral cavity disorders by local intra-oral forms of drug delivery.

### BACKGROUND OF THE INVENTION

Acids in the oral cavity have an erosive effect on teeth. The source of acids in the oral cavity could be the intake of acidic foods and drinks in otherwise healthy oral cavity, reflux of acid generated in the gastric cavity to the oral cavity, or the fermentation of food debris by bacteria that colonies the dental surface (dental plaque). Acids are erosive to teeth and tooth enamel, the outer layer, will be attacked first before damage to the inner layer, the dentin.

In one study, enamel demineralization in rats was linked to the hydrogen-ion (pH) when a 1- to 1.5-percent hydrogen peroxide solution was added to their drinking water (Shapiro, M., V. Brat, and B. H. Ershoff, “Induction of Dental Caries and Pathological Changes in Periodontium of Rat with Hydrogen Peroxide and Other Oxidizing Agents,” Journal of Dental Research, 39:332-343, 1960).

In another publication, the erosive effect of wine on tooth enamel was related to its acidity (Meurman J H, Vesterinen M., “Wine, alcohol, and oral health, with special emphasis on dental erosion”, Quintessence Int. 2000 November-December;31(10):729-33).

Furthermore, and in view of the increasingly documented incidence of tooth erosion caused by acidic soft drinks, the dental erosive effect of some acidic soft drinks was demonstrated in-vitro (West N X, Hughes J A, Addy M. “The effect of pH on the erosion of dentine and enamel by dietary acids in vitro”, J Oral Rehabil. 2001 September;28(9): 860-4).

Similarly, the dental erosive effect of some brands of beer was demonstrated in-vitro and related to their acidity (Nogueira F N, Souza D N, Nicolau J., “In vitro approach to evaluate potential harmful effects of beer on teeth”, J Dent. 2000 May;28(4):271-6).

Sugars derived from fruits are fermented to acids by the oral microflora and the intra-oral acidogenic potential of fruit consumption was shown by Beighton et.al. (D. Beighton, S. R. Brailsford, S. C. Gilbert, D. T. Clark, S. Rao, J. C. Wilkins, E. Tarelli, K. A. Homer, “Intra-Oral Acid Production Associated with Eating Whole or Pulped Raw Fruits”, Caries Research 2004;38:341-349).

Etiological factors for the cascade of dental plaque formation, dental caries, gingivitis, periodetitis and related diseases are poor oral hygiene. and erosive damage to tooth enamel.

On the other hand, the secretion of saliva is considered an important defence mechanism. Saliva contains a number of minerals (including calcium and fluoride) and other substances that help to protect teeth by lowering mouth acidity. Chewing causes more saliva to be produced, and health authorities in some countries advocate chewing sugar-free gum for 20 minutes after a meal, to reduce the risk of dental decay, as it stimulates saliva production.

Oral hygiene can be maintained by mechanical procedures (brushing, dental floss, wedges and the like) and by the administration of chemicals to the oral cavity (dentifrice, liquid dentifrice, mouth rinse preparations, antibiotics, and other drugs).

The regular use of toothpaste with fluoride is recommended as there is an established body of evidence that supports their protective effect on the surface of the teeth against dental plaque formation and related consequences.

Dental plaque is made up of bacteria, like Actinomyces species, Streptococcus sanguis, S. mutans, and other Streptococcus species, as well as extracellular matrix synthesized by the bacteria. The extracellular matrix provides plaque organisms with strong adhesive and cohesive properties that allows plaque to adhere to the surfaces of oral structures. Unlike debris, dental plaque cannot be removed by flushing the mouth with fluids.

Plaque formation is considered an etiologic factor in the evolvement of dental caries, gingivitis, and periodontal disease.

The pathogenic potential of dental plaque depends on its microbial composition, including the metabolic products of microbes, dietary patterns, and the intrinsic resistance of the host comprised by the strength and entirety of teeth enamel.

Mechanical plaque control methods have the potential to maintain adequate levels of oral hygiene. Ideally, teeth should be brushed after every meal and snack/drink. However this is difficult to do, and clinical experience and population-based studies demonstrate that such methods, even once daily teeth brushing, are not being employed sufficiently by large numbers of the population. The need for additional help in controlling bacterial plaque provides the rationale for chemical adjuncts to the mechanical oral hygiene regimens.

Oral hygiene finger devices configured to clean the teeth and gums of a user were described recently in U.S. Pat. Nos. 6,647,549, and 6,721,987 to McDevitt et al.

The devices described in McDevitt et al. are very limited as drug delivery devices because of the limitation of the materials used to manufacture the devices.

Furthermore, these devices are used to clean teeth and gums and such a cleaning process will be few minutes long at most, and consequently the therapeutic effect of additives delivered from these devices will be short-lived, Furthermore, the addition of anti-ulcer agents to the device as described in McDevitt et al. were aimed to treat peptic ulcer disease, as stated in the description and claims of McDevitt et al.

Therefore, it would be desirable to provide improved methods for prevention or treatment of oral cavity disorders.

### SUMMARY OF THE INVENTION

The present invention relates to a method for treatment or prevention of oral cavity diseases and disorders and/or related states, in a subject in need of such treatment, comprising local administration of antisecretory drugs and/or antacids, in acceptable pharmacological forms and pharmaceutical formulations.

In the context of the present invention, the term “local intra-oral administration” refers to administration of a pharmaceutical composition to the oral cavity, where it remains until dissolution of its active ingredients or cessation of its activity. This term includes sublingual administration, flushing of the oral cavity, intra oral sustained release systems, toothpaste based release systems, temporary and permanent dental restorative materials based release systems, and the like.

The present invention relates to a method for prevention or treatment of oral cavity disorders by local intra-oral forms of drug delivery. The method of the present invention allows for the effective therapy of dental plaques, dental caries, gingivitis, periodontitis and associated diseases by the local intra-oral delivery of antacids and antisecretory drugs currently used for the treatment of peptic ulcer disease or gastro-esophageal reflux disease.

According to preferred embodiments of the present invention, the oral cavity diseases and disorders and/or related states which are treated by the inventive method are described in relation to the oral cavity of a healthy individual in need of alternative oral hygiene procedures or augmentation of conventional oral hygiene methods.

Further according to preferred embodiments of the present invention, the oral cavity diseases and disorders and/or related states which are treated by the present invention are those selected from the group including, but not limited to: dental caries, gingivitis, periodontal pockets, stomatitis, bacterial infections of oral cavity tissues and structures, fungal infections of oral cavity tissues and structures, viral infections of oral cavity tissues and structures, inflammatory disorders of oral cavity tissues and structures, proliferative disorders of oral cavity tissues and structures, and infiltrative disorders of oral cavity tissues and structures.

Still further according to preferred embodiments of the present invention, the oral cavity diseases and disorders and/or related states which are treated by the present invention are those selected from the group including, but not limited to: oral cavity of intubated patients for artificial respiration, oral cavity of patients hospitalized in the intensive care units and like, oral cavity of patients undergoing surgery under general anesthesia, oral cavity of unconscious and comatose patients, oral cavity of paralyzed and disabled patients, oral cavity of patients breathing through tracheotomy or tracheostomy, and oral cavity of patients following chemical or thermal injury to head and neck area.

Additionally according to preferred embodiments of the present invention, the oral cavity diseases and disorders and/or related states which are treated by the present invention are those selected from the group including, but not limited to: gastroesophageal reflux disease, peptic ulcer disease and helicobacter pylori related disease.

Moreover according to preferred embodiments of the present invention, the antisecretory drugs and antacids are those selected from the group including, but not limited to: H2-receptor antagonists, proton pump inhibitors, antacids, or any agent capable of either decrease the release of proton ions (H+) into or capturing the proton ions in the oral cavity, or their combinations.

Further according to preferred embodiments of the present invention, the H2-receptor antagonists are those selected from the group including, but not limited to: cimetidine, ranitidine, and famotidine.

Still further according to preferred embodiments of the present invention, the proton pump inhibitors are those selected from the group including, but not limited to: omeprazole, esomeprazole, lansoprazole and pantoprazole.

Additionally according to preferred embodiments of the present invention, the antacids are those selected from the group including, but not limited to: aluminum hydroxide, magnesium hydroxide, bismuth and sucralfate.

Moreover according to preferred embodiments of the present invention, the agents capable of either decreasing the release of proton ions (H+) into the oral cavity or capturing the proton ions in the oral cavity are those selected from the group including, but not limited to: florides, indomethacin and benzoate.

Further according to preferred embodiments of the present invention, local administration strategies are those selected from the group including, but not limited to, intra oral sustained release systems, toothpaste-based release systems, temporary and permanent dental restorative material-based release systems, temporary and permanent micro- and nano-particle laden polymers, temporary and permanent polymers with chemical or covalent bonds to drugs, orthodontic device-based drug release systems, prosthetic element-based drug release systems, denture and crown-based drug release systems, sublingual tablets, sublingual capsules, mouthwash-based drug release systems, dental floss-based drug release systems, dental wedge-based drug release systems, dental implant-based drug release systems, surgical devices and suture-based drug release systems, periodontal device-based drug release systems, chewing gum-based drug release systems, candy and like-based drug release systems, sucking and like-based drug release systems, dipping and like-based drug release systems, inhalators, and sprays.

Still further according to preferred embodiments of the present invention, the drug release systems are those selected from the group including, but not limited to, materials coated by drugs, materials sprayed by drugs, materials soaked in drugs, materials laden with drugs, materials laden with encapsulated drugs, drug micro- and nano-particles incorporated into the materials during the manufacturing process of these materials, materials with covalent or chemical bonds to drugs.

Additionally according to preferred embodiments of the present invention, the local administration strategy is over-night usage of temporary crowns or full/partial dentures, manufactured from or coated by drug-laden materials or materials with chemical or covalent bonds to drugs.

Moreover according to preferred embodiments of the present invention, the local administration strategy is tubes and airways and other parts used in artificial respiration, manufactured from or coated by drug-laden materials or materials with chemical or covalent bonds to drugs.

Further according to preferred embodiments of the present invention, the local administration strategy is nasal or oral gastric tubes, used for gastrointestinal tract evacuation or feeding, manufactured from or coated by drug-laden materials or materials with chemical or covalent bonds to drugs.

Still further according to preferred embodiments of the present invention, the acceptable pharmacological forms and pharmaceutical formulations are those selected from the group including, but not limited to, acceptable salts, drug derivatives, pro-drugs, co-drugs, and acceptable vehicles.

Moreover according to preferred embodiments of the present invention, the acceptable pharmacological forms and pharmaceutical formulations are those selected from the group including, but not limited to, aqueous solution, non-aqueous solution, alcoholic solution, mixed aqueous-alcoholic solution, mineral oil, glycerol, and polyethylene glycol.

Further according to preferred embodiments of the present invention, the acceptable pharmacological forms and pharmaceutical formulations are those selected from the group including, but not limited to, lotions, sprays, suspensions, ointments, gels, hydrogels, viscosified solution systems, gelatin, gel-forming systems, liposomes, emulsions, micro- and nano-particle laden polymers.

Still further according to preferred embodiments of the present invention, the acceptable pharmacological forms and pharmaceutical formulations are those selected from the group including, but not limited to, membrane-bound devices, drug laden cellulose derivatives, drug laden strips, drug laden fibers, drug laden textile, and drug laden collagens.

Moreover according to preferred embodiments of the present invention, the subject in need of treatment is a human, animal, or pet.

It is thus appreciated that the method of the present invention will have far-reaching consequences in the field of medical treatment of oral cavity diseases.

### DETAILED DESCRIPTION OF THE INVENTION

Surprisingly, Proton pump inhibitors and Histamine H2-receptor antagonists have been found by the inventor of the present invention to be effective in the local prevention and treatment of oral cavity diseases, particularly in the prevention or treatment of gingivitis and associated diseases.

Proton pump inhibitors and Histamine H2 receptor antagonists have been proved to be very effective for suppressing intragastric acidity. In general, H2-receptor antagonists are considered to exert their effects on gastric acid secretion more rapidly than proton pump antagonists.

H2-receptor antagonist interaction with the histamine H2-receptor leads to inhibition of histamine-stimulated cAMP production, and consequently to inhibition of extracellular proton ion secretion.

Different proton pump inhibitors bind at different sites in the intracellular proton transport pathway and the covalent binding to H+, K+ ATPase lead to proton pump inhibition, and consequently to inhibition of proton ion secretion to the extracellular space.

Examples of gastric antacids include aluminum hydroxide and magnesium hydroxide.

Sucralfate produces a coating over the gastric mucosa and thus protects mucosa from attack by acid. Sucralfate also binds proton-ions and suppresses growth of helicobacter pylori. Gastric antisecratory drugs include histamine H2-receptor antagonists (also known as H2-blockers) and proton-pump inhibitors (PPI's).

H2-receptor antagonists include cimetidine, ranitidine and famotidine. In some countries low-dose cimetidine and ranitidine are available as over-the-counter (OTC) drugs. Proton-pump inhibitors include omeprazole, esomeprazole (the optical isomer of omeprazole), lansoprazole and pantoprazole.

Gastric antisecretors were shown to be effective for suppressing intragastric acidity also in healthy individuals (Hamilton M I, Sercombe J, Pounder R E, “Decrease of intragastric acidity in healthy subjects dosed with ranitidine 75 mg, cimetidine 200 mg, or placebo”, Dig Dis Sci. 2002 January;47(1):54-7).

Following rapid degradation of sugar by oral bacteria to acidic end-products, the pH value of their immediate environment can fall to levels where glycolysis and growth cease (Phan T N, Nguyen P T, Abranches J, Marquis R E, “Fluoride and organic weak acids act as respiration inhibitors for oral streptococci in acidified environments”, Oral Microbiol Immunol. 2002 April;17(2):119-24; Iwami Y, Guha-Chowdhury N, Yamada T, “Mechanism of inhibition of acid production in Streptococcus mutans by sodium ions under strictly anaerobic conditions”, Oral Microbiol Immunol. 1997 June; 12(3):178-82).

At this point one of the mechanisms that oral bacteria can use to protect themselves from harmful cytoplasmic acidification is by pumping protons out of the cells (Quivey R G Jr, Kuhnert W L, Hahn K, “Adaptation of oral streptococci to low pH”, Adv Microb Physiol. 2000;42:239-74).

Prior art research, such as the studies about to be described, could not provide a solid and convincing case for the local intra-oral usage of antacids and gastric antisecretory drugs as a therapy for oral cavity diseases.

However, with the unexpected results obtained by the inventor of the present invention, it can be concluded that antacids and gastric antisecretory drugs could well be used as an efficient and effective method of local intra-oral prevention or treatment of oral cavity diseases and disorders, particulary dental plaques, dental caries, gingivitis, periodontitis and related diseases.

In the prior art there is a paucity of knowledge about the possible mechanisms of action of proton pump inhibitors and H2-receptor antagonists on bacterial growth and metabolism.

In one study, the inhibition of proton-translocating membrane-ATPases of oral bacteria will disallow them from using this survival mechanism, and consequently prevent bacterial survival when excessive fermentation turns bacterial cytoplasm very acidic (Cotter PD, Hill C, “Surviving the acid test: responses of gram-positive bacteria to low pH”, Microbiol Mol Biol Rev. 2003 September;67(3):429-53).

In another study, the proton-translocating membrane ATPases of oral streptococci was reported to be sensitive to the inhibitors dicyclohexylcarbodiimide (DCCD) and azide, but insensitive to ouabain and vanadate (Sutton S V, Marquis R E,“Membrane-associated and solubilized ATPases of Streptococcus mutans and Streptococcus sanguis”, J Dent Res. 1987 June;66(6):1095-8).

Although proton-pump inhibitors that are currently used as gastric antisecretory agents potentially could inhibit proton-translocating membrane-ATPases of oral streptococci, this inhibitory effect is not supported by published experimental evidence.

In another study, the inhibitory influence of histamine H2-antagonists on the growth of mycobacteria was described in a report from 1990 (Meindl W, Friese-Kimmel A, Lachenmayr F, Buschauer A, Schunack W., “The effect of agonists and antagonists of histamine H1- and H2-receptors on the growth of Mycobacterium tuberculosis”, H 37 Ra. Arch Pharm (Weinheim). 1990 May;323(5):267-72).

Yet in another study, a new anti-Helicobacter pylori (H. pylori) agents that possess H2-antagonist properties were reported (Sorba G, Bertinaria M, Di Stilo A, Gasco A, Scaltrito M M, Brenciaglia M I, Dubini F., “Anti-Helicobacter pylori agents endowed with H2-antagonist properties”, Bioorg Med Chem Lett. 2001 Feb. 12;11(3):403-6).

These and other studies have revealed only some of the characteristics of the potential interaction of proton-pump inhibitors and histamine H2-receptor antagonists with oral bacteria.

The possibility that histamine H2-receptor antagonists that are currently used as gastric antisecretors could inhibit bacterial growth was not addressed in the literature.

Prior art finger devices for cleaning the teeth and gums were described earlier.

Embodiments of the invention include a finger glove that can fit onto a human finger, or an oral cleaning device or dental wipe also designed to fit a human finger. Various additives can be applied to both devices, and in some embodiments anti-ulcer agents were suggested as such additives. In one embodiment bismuth salts were suggested as additive. Other suggested anti-ulcer additives were tetracycline, erythromycin, clarithromycin, omeprazole, metronidazole, or other antibiotics. Furthermore, any additive useful for treating peptic ulcers, such as H2-blockers, sucralfate, and the like, were suggested as well. In another embodiment, the texturized surface of the cleaning device has been treated with an anti-ulcer composition.

As can be appreciated, the prior art devices previously described which are used to clean teeth and gums will have a therapeutic effect for few minutes at most, as compared to embodiments of the present invention where local drug delivery to the oral cavity can be done through sophisticated sustained release systems that will allow the delivery of sufficient therapeutic concentrations of the suggested drugs throughout a 24 hours day and night cycle.

The McDevitt et al. anti-ulcer agents previously described were added for treatment of peptic ulcer disease, whereas in the present invention, the inventor surprisingly found that anti-ulcer agents are effective in local treatment of oral cavity diseases.

### EXAMPLES

The following examples are presented to illustrate various aspects of the present invention.

### Example 1

A 40 year-old-male was suffering from accelerated cariogenicity and progressive gingivitis in recent years despite repeated periodontal, restorative, and prophylactic treatments. His general medical history included recent symptoms that were related to peptic ulcer. Despite negative gastroscopy and negative test for Helicobacter pylori, a H2-antagonist treatment (Famotidine 40 mg per day) was prescribed. Half year later, epigastric symptoms were relieved but symptoms of reflux persisted. Reflux symptoms were relieved later when the patient started to take famotidine tablets sublingually instead of swallowing the tablets. In the following months the gingivitis improved dramatically, and the patient was consulted to continue with the sublingual famotidine treatment. Twice a year dental follow-ups in the next 5 years under sublingual famotidine treatment were unremarkable and new caries were not detected.

### Example 2

An ongoing study is now being carried out on healthy young (25-40 years old) volunteers. The oral cavity pH is measured 1, 2, and 3 hours after a meal and at wakeup in the morning. These measurements are done daily for each participant, 3 weeks without treatment, 3 weeks under sublingual famotidine (40 mg per day), and 3 weeks under p.o. famotidine (40 mg per day).

Preliminary results showed that the rank order of the highest oral cavity pH was: Sublingual Famotidine treatment>p.o. Famotidine treatment>no treatment.

### Example 3

Twelve male participants (24-42 years old) suffering from gingivitis were divided into 4 groups. Participants of the first group (No.=3) were treated by conventional oral hygiene procedures, including mechanical procedures and usage of mouthwash preparations for 3 months. Participants of the second group (No. =3) were treated by swallowing tablet of Famotidine 40 mg, once daily at bed time, for 3 months. Participants of the third group (No.=3) were treated by sublingual tablet of Famotidine 40 mg, once daily at bed time, for 3 months. Participants of the fourth group (No.=3) were treated by sublingual tablet of Omeprazole 20 mg, once daily at bed time, for 3 months. All participants were screened for dental caries and treated accordingly.

The screening revealed no significant inter-group difference in the prevalence of dental caries. There was also no significant inter-group difference in the severity of gingivitis at recruitment. Each participant was examined at recruitment and after then bi-weekly up to 3 months from recruitment (7 examinations for each participant). At each visit gingival index was determined using a standard Loe Silness gingival index (GI) chart, which is accessible at the following website: http://www.medal.org/sheets/ch9/gingival %20index %20Loe %20Silness.xls.

The GI scores of examination No.7 (at 3 months from recruitment) compared to GI scores at examination No.1 (at recruitment), were calculated as mean GI reduction for each group. Mean GI reduction was 16.3% for the first group, 17.1% for the second group, 31% for the third group and 27.5% for the fourth group.

Each example described above is provided by way of explanation, not limitation of the invention. It will be apparent to those of ordinary skill in the art that various modifications and variations can be made in the present invention without departing from the scope or spirit of the invention. For instance, features illustrated or described as part of one embodiment, can be used on another embodiment to yield a still further embodiment. Thus, it is intended that the present invention covers such modifications and variations as come within the scope of the appended claim and its equivalents.

1. A method for treatment or prevention of oral cavity diseases and related states, in a subject in need of such treatment, comprising the administration of local intra-oral drug delivery systems for the release of a pharmaceutical composition having as the active ingredient at least one member of the group consisting of an antisecretory drug and an antacid.

2. The method of claim 1, wherein said oral cavity diseases and related states is the oral cavity of healthy individual in need of alternative oral hygiene procedure or augmentation of conventional oral hygiene method.

3. The method of claim 1, wherein said oral cavity diseases and related states are those selected from the group consisting of: dental caries, gingivitis, periodontal pockets, stomatitis, bacterial infections of oral cavity tissues and structures, fungal infections of oral cavity tissues and structures, viral infections of oral cavity tissues and structures, inflammatory disorders of oral cavity tissues and structures, proliferative disorders of oral cavity tissues and structures, and infiltrative disorders of oral cavity tissues and structures.

4. The method of claim 1, wherein said oral cavity diseases and related states are those selected from the group consisting of: oral cavity of intubated patients for artificial respiration, oral cavity of patients hospitalized in the intensive care units and like, oral cavity of patients undergoing surgery under general anesthesia, oral cavity of unconscious and comatose patients, oral cavity of paralyzed and disabled patients, oral cavity of patients breathing through tracheotomy or tracheostomy, and oral cavity of patients following chemical or thermal injury to head and neck area.

5. The method of claim 1, wherein said oral cavity diseases and related states are those selected from the group consisting of: gastroesophageal reflux disease, peptic ulcer disease and helicobacter pylori related disease.

6. The method of claim 1, wherein said antisecretory drugs and antacids are those selected from the group consisting of: H2-receptor antagonists, proton pump inhibitors, antacids, or any agent capable of either decrease the release of proton ions (H+) into or capturing the proton ions in the oral cavity, or their combinations.

7. The method of claim 6 wherein said H2-receptor antagonists are those selected from the group consisting of: cimetidine, ranitidine, famotidine, and combinations thereof.

8. The method of claim 6 wherein said proton pump inhibitors are those selected from the group consisting of: omeprazole, esomeprazole, lansoprazole, pantoprazole, and combinations thereof.

9. The method of claim 6 wherein said antacids are those selected from the group consisting of: aluminum hydroxide, magnesium hydroxide, bismuth, sucralfate, and combinations thereof.

10. The method of claim 6 wherein said agent capable of either decrease the release of proton ions (H+) into or capturing the proton ions in the oral cavity are those selected from the group consisting of: florides, indomethacin and benzoate, and combinations thereof.

11. The method of claim 1, wherein said local intra-oral drug delivery systems are those selected from the group consisting of: intra oral sustained drug release systems, toothpaste based drug release systems, temporary and permanent dental restorative materials based drug release systems, temporary and permanent micro- and nano-particle laden polymers, temporary and permanent polymers with chemical or covalent bonds to drugs, orthodontic devices based drug release systems, prosthetic elements based drug release systems, denture and crowns based drug release systems, sublingual tablets, sublingual capsules, mouthwash based drug release systems, dental floss based drug release systems, dental wedges based drug release systems, dental implants based drug release systems, surgical devices and sutures based drug release systems, periodontal devices based drug release systems, chewing gum based drug release systems, candy and like based drug release systems, sucking and like based drug release systems, dipping and like based drug release systems, inhalators, and sprays.

12. The method of claim 1, wherein said drug delivery systems are those selected from the group consisting of: materials coated by drugs, materials sprayed by drugs, materials soaked in drugs, materials laden with drugs, materials laden with encapsulated drugs, drug micro- and nano-particles incorporated into the materials during the manufacturing process of these materials, materials with covalent or chemical bonds to drugs.

13. The method of claim 1, wherein said local intra-oral drug delivery systems is over-night usage of temporary crowns or full/partial dentures, manufactured from or coated by drug-laden materials or materials with chemical or covalent bonds to drugs.

14. The method of claim 1, wherein said local intra-oral drug delivery systems is tubes and airways and other parts used in artificial respiration, manufactured from or coated by drug-laden materials or materials with chemical or covalent bonds to drugs.

15. The method of claim 1, wherein said local intra-oral drug delivery systems is nasal or oral tubes and devices, used for gastrointestinal tract examination, evacuation or feeding, manufactured from or coated by drug-laden materials or materials with chemical or covalent bonds to drugs.

16. The method of claim 1, wherein said pharmaceutical composition are those selected from the group consisting of: acceptable salts, drug derivatives, pro-drugs, co-drugs, and acceptable vehicles.

17. The method of claim 1, wherein said pharmaceutical composition are those selected from the group consisting of: aqueous solution, non-aqueous solution, alcoholic solution, mixed aqueous-alcoholic solution, mineral oil, glycerol, and polyethylene glycol.

18. The method of claim 1, wherein said pharmaceutical composition are those selected from the group consisting of: lotions, sprays, suspensions, ointments, gels, hydrogels, viscosified solution systems, gelatin, gel-forming systems, liposomes, emulsions, micro- and nano-particle laden polymers.

19. The method of claim 1, wherein said pharmaceutical composition are those selected from the group consisting of: membrane-bound devices, drug laden cellulose derivatives, drug laden strips, drug laden fibers, drug laden textile, and drug laden collagens.

20. The method of claim 1, wherein said a subject in need of treatment is a human, animal, or pet.
